(19) United States
(12) Patent Application Publication
(10) Pub. No.: US 2015/0282774 A1
(43) Pub. Date: Oct. 8, 2015

(54) STATIONARY GANTRY COMPUTED TOMOGRAPHY SYSTEMS AND METHODS WITH DISTRIBUTED X-RAY SOURCE ARRAYS

(71) Applicant: The University of North Carolina at Chapel Hill, Chapel Hill, NC (US)

(72) Inventors: Yueh Z. Lee, Chapel Hill, NC (US); Jing Shan, Chapel Hill (CN); Otto Z. Zhou, Chapel Hill, NC (US); Jianping Lu, Chapel Hill, NC (US)

(73) Assignee: The University of North Carolina at Chapel Hill, Chapel Hill, NC (US)

(21) Appl. No.: 13/970,134

(22) Filed: Aug. 19, 2013

Related U.S. Application Data
(60) Provisional application No. 61/684,575, filed on Aug. 17, 2012.

(51) Int. Cl. A61B 6/00; A61B 6/03

(52) U.S. Cl. CPC A61B 6/4435; A61B 6/4405; A61B 6/032; A61B 6/4014; A61B 6/482

(57) ABSTRACT

Systems and methods for x-ray imaging are disclosed, particularly non-rotating, stationary gantry and mobile x-ray computed tomography systems and methods for imaging a subject, and particularly for imaging the head, spine, and neck of a subject. Compared to rotating-gantry computed tomography scanners, non-rotating stationary gantry x-ray computed tomography scanners are more mobile and transportable. Non-rotating stationary gantry x-ray computed tomography scanners can thus be used in mobile transport units and in-field applications.

### CROSS REFERENCE TO RELATED APPLICATION

This application claims the benefit of and priority to U.S. Provisional Patent Application Ser. No. 61/684,575, filed Aug. 17, 2012, the disclosure of which is incorporated herein by reference in its entirety.

### GOVERNMENT INTEREST

This invention was made with government support under Grant Nos. R01CA134598 and U54CA119343 awarded by The National Institutes of Health. The government has certain rights in the invention.

### TECHNICAL FIELD

The subject matter disclosed herein relates generally to systems and methods for x-ray imaging. More particularly, the subject matter disclosed herein relates to non-rotating, stationary gantry and mobile x-ray computed tomography systems and methods for imaging a subject, and particularly for imaging the head, spine, and neck of a subject.

### BACKGROUND

X-ray imaging is widely used in many areas including medical diagnostics and treatment, industrial inspection and testing, security screening and detections. One form of x-ray imaging, non-contrast computed tomography (CT), is useful in a number of medical applications, including for example diagnosing ischemic stroke. Every 40 seconds, someone in the United States has a stroke. Over 795,000 strokes occur a year, resulting in direct and indirect health care costs that exceed 41 billion dollars per year.

Current CT scanners are large complex devices that are not portable or readily mobile. As such, a patient in need of a CT scan must be transported to the nearest CT scanner, sometimes at a great distance. For some medical conditions, such as ischemic stroke, early diagnosis and treatment is vitally important in improving morbidity and mortality. Unfortunately, the window of time for optimal treatment can be significantly reduced by the transit time to a location, hospital or medical facility equipped with a CT scanner.

It would therefore be beneficial to bring the imaging system to the patient, thereby potentially reducing the time to treatment and improving patient outcome. Thus, it is desirable to have CT systems and methods that are portable and mobile such that they can be readily brought to a patient in need of a CT scan.

Current CT scanners include a rotating gantry design where the x-ray tube and detector pair rotate around the patient in a circular motion to generate the projection views needed for CT reconstruction. A non-rotating stationary gantry CT system would reduce the mechanical complexity thereby making the device or system more portable. Upkeep and maintenance of such a system could also be reduced. Previous attempts at designing a non-rotating gantry CT, such as the Dynamic Spatial Reconstructor (DSR) and the electron-beam CT (EBCT), have been met with limited success. The cost, size, complexity and maintenance related issues of the DSR and EBCT systems have generally been prohibitive.

Therefore, there remains an unmet need for mobile x-ray CT systems and methods for imaging a subject, and particularly for imaging the head, spine, and neck of a subject.

### SUMMARY

In accordance with this disclosure, non-rotating stationary gantry and mobile x-ray computed tomography systems and methods for imaging a subject, and particularly for imaging the head, spine, and neck of a subject, are provided.

In one aspect, a non-rotating stationary gantry and mobile x-ray computed tomography imaging system is provided. The imaging system can comprise one or more spatially distributed x-ray source arrays for emitting x-ray radiation, one or more x-ray detector arrays for detecting the x-ray radiation from the one or more x-ray source arrays, and an electronic control for controlling x-rays from individual x-ray focus spots of the one or more spatially distributed x-ray source arrays with programmable photon flux and pulse sequence for synchronization of x-ray exposure with data collection of the one or more x-ray detector arrays.

Although some of the aspects of the subject matter disclosed herein have been stated hereinabove, and which are achieved in whole or in part by the presently disclosed subject matter, other aspects will become evident as the description proceeds when taken in connection with the accompanying drawings as best described hereinbelow.

### BRIEF DESCRIPTION OF THE DRAWINGS

The features and advantages of the present subject matter will be more readily understood from the following detailed description which should be read in conjunction with the accompanying drawings that are given merely by way of explanatory and non-limiting example, and in which:

FIG. 1 is a block diagram of an exemplary system for obtaining a multi-beam image of an object according to one aspect of the subject matter described herein;

FIG. 2 is a block diagram of an exemplary system for obtaining a multi-projection image of an object according to another aspect of the subject matter described herein;

FIG. 3 is a schematic, cross-section side view of a multi-pixel field emission x-ray source according to an aspect of the subject matter described herein;

FIG. 4A is a perspective view of an x-ray imaging system including an x-ray generator device for applying binary multiplexing x-ray radiography to an object according to an embodiment of the subject matter described herein;

FIGS. 4B and 4C are graphs of an example of pulsed current applied to the x-ray pixels and the generated x-ray intensities over a period of time for generating pulsed x-ray radiation;

FIGS. 5A-5C are schematic diagrams of a conventional scanning sequential imaging system that is imaging an object in sequence from different projection angles;

FIGS. 6A-6C are schematic diagrams of an exemplary BMXR system that is imaging an object in accordance with the subject matter described herein;

FIG. 7 is a flow chart of an exemplary process of binary multiplexing x-ray radiography according to an embodiment of the subject matter described herein;

FIG. 8 is a schematic diagram of an exemplary BMXR system operable to generate multiplexed composite x-ray beams including signals based on the predetermined Hadamard binary transform and irradiate an object with the composite x-ray beams according to an embodiment of the subject matter described herein;

FIG. 9 is a schematic diagram of an exemplary CT imaging system having multi-beam field emission pixels according to an embodiment of the subject matter described herein;

FIG. 10 is a schematic diagram of a conventional rotational-gantry CT scanning device;

FIG. 11 is an illustration of a conventional rotational-gantry CT scanning device;

FIG. 12 is a schematic diagram of an exemplary CT scanning system having a non-rotational stationary gantry design according to an embodiment of the subject matter described herein;

FIG. 13 is a schematic diagram of an exemplary CT scanning system having a non-rotational stationary gantry design according to an embodiment of the subject matter described herein; and

FIG. 14 is an illustration of a reconstructed single slice from the stationary head CT mockup of the ACR accreditation phantom, acquired at significantly reduced dose.

### DETAILED DESCRIPTION

The subject matter disclosed herein is directed to portable, non-rotating stationary gantry computed tomography (CT) scanners and methods. A CT scanner as disclosed herein can in some embodiments utilize one or more spatially distributed x-ray source arrays to generate the projection images needed for CT reconstruction. In some embodiments, a CT scanner can be designed specifically for imaging of head, spine and neck for mobile applications. In some aspects, a CT scanner can be equipped with wireless communication capability for transmitting the images to one or more centralized or desired location(s) for analysis by a medical professional.

In some embodiments, the subject matter disclosed herein is directed to multiplexing x-ray radiographic techniques, devices and methods that utilize a multi-beam x-ray source, an x-ray detector, and binary transform techniques. The radiographic techniques disclosed herein according to one aspect are referred to as binary multiplexing x-ray radiography (BMXR), such as disclosed in U.S. patent application Ser. No. 11/804,897, now U.S. Pat. No. 8,189,893, which is incorporated herein by reference in its entirety. In accordance with the BMXR techniques disclosed herein, during the data collection process, an on/off state (also referred to as the “binary state”) of a multi-beam x-ray source follows the form of a predetermined binary transform. The on-off states of the x-ray source can generate x-ray beams including signals based on the predetermined binary transform. An object, or in some embodiments a subject such as a patient, can be irradiated with the generated x-ray beams. After irradiation of an object, transmitted or fluorescent x-ray beams can be detected by an x-ray detector which records x-ray intensities of the multiplexing x-ray signals corresponding to the binary states of the x-ray source. The recorded x-ray intensity data can then be processed through an inverse binary transform to recover the original x-ray signals generated from each beam of the multi-beam x-ray source.

BMXR enables many new x-ray imaging and x-ray analysis applications. By using different forms of binary transforms, BMXR can reduce data collection time, enhance signal-to-noise ratio (SNR), and provide better power distribution of an x-ray source in digital radiography and fluorescence spectroscopy. By use of a multi-beam x-ray source, BMXR can allow parallel imaging/spectroscopy analysis of an object from multiple x-ray beams simultaneously using a single detector. BMXR can enhance the imaging speed in computed tomography (CT), tomosynthesis, fluoroscopy, angiography, multi-energy radiography, and x-ray fluorescence spectroscopy analysis. Suitable applications of BMXR include, for example, medical diagnostics and treatment, industrial non-destructive testing (NDT) and x-ray fluorescence (XRF) analysis, and security screening and detections.

As referred to herein, the term “nano-structured” or “nanostructure” material designates materials including nanoparticles with particle sizes less than 100 nm, such as nanotubes (e.g.—carbon nanotubes). These types of materials have been shown to exhibit certain properties that have raised interest in a variety of applications.

As referred to herein, the term “multi-beam x-ray source” designates devices that can simultaneously generate multiple x-ray beams. For example, the “multi-beam x-ray source” can include a field emission based multi-beam x-ray source having electron field emitters. The electron field emitters can include nanostructure based materials.

As referred to herein, the term “binary transform” refers to the concept of multiplexing techniques, including Hadamard transforms and other suitable binary transforms. Generally, the binary transform can be presented by a binary transform matrix whose elements are either 1 or 0, which represents the on or off state of the signal source, respectively. Binary transforms, such as Hadamard transforms, can be applied to various kinds of applications including microscopy, optical spectroscopy, mass spectrometry, and magnetic resonance imaging (MRI).

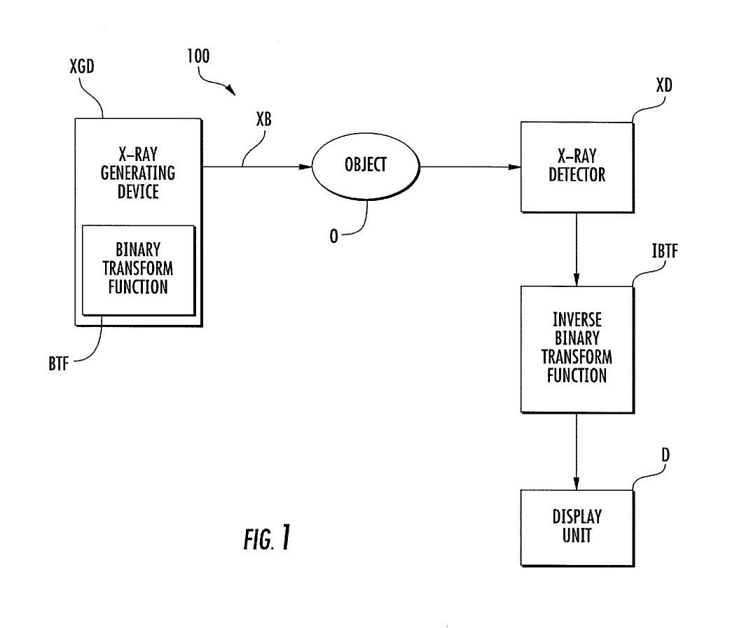

FIG. 1 is a block diagram of an exemplary BMXR system generally designated 100 for obtaining a multi-beam image of an object according to one aspect of the subject matter described herein. Referring to FIG. 1, a binary transform function BTF can control an x-ray generating device XGD having multiple pixels to generate multiple, composite x-ray beams XB including signals based on a predetermined binary transform and configured to direct x-ray signals XS toward object O for irradiating the object. In this example, x-ray beams XB are substantially projected towards object O from a single direction. Further, in this example, each of x-ray beams XB have different x-ray energy spectra. Different x-ray energy spectra can be achieved, for example, by using different anode KVp or different anode materials for the different x-ray beam pixels. In this way, BMXR can provide for fast imaging in energy subtraction radiography imaging and multi-energy monochromatic imaging.

For monochromatic imaging, a system in accordance with the subject matter described herein can include a monochromator configured to generate a monochromatic x-ray beam for imaging an object. The monochromator can generate multiple monochromatic x-ray beams having either the same or different x-ray energies for monochromatic x-ray imaging.

The binary state of the signals of x-ray beams XB can be based on a predetermined binary transform. Particularly, the signals can follow the form of a predetermined binary transform matrix. The binary state of the signals of the x-ray beams can be based on a pattern of 0 and 1 elements in the predetermined binary transform matrix.

After passing through object O, x-ray beams XB can be detected by an x-ray detector XD, such as for example a high frame rate x-ray detector XD. X-ray detector XD can continuously capture the composite x-ray beams XB. After all or at least a portion of x-ray beams XB are collected and stored as x-ray signal data in a memory, an inverse binary transform function IBTF can apply an inverse binary transform to the stored x-ray signal data to recover the original set of generated signals.

Using the same principle of binary transform technique, BMXR in accordance with the subject matter described herein can be used to obtain multi-projection images of an object from multiple x-ray sources simultaneously using a single detector. This imaging technique can enhance the imaging speed in CT, tomosynthesis, fluoroscopy (e.g., digital fluoroscopy), angiography, and multi-energy radiography. Further, this imaging technique can lead to enhanced detection speed in industrial applications such as non-destructive testing (NDT), x-ray fluoroscopy (XRF), and diffraction.

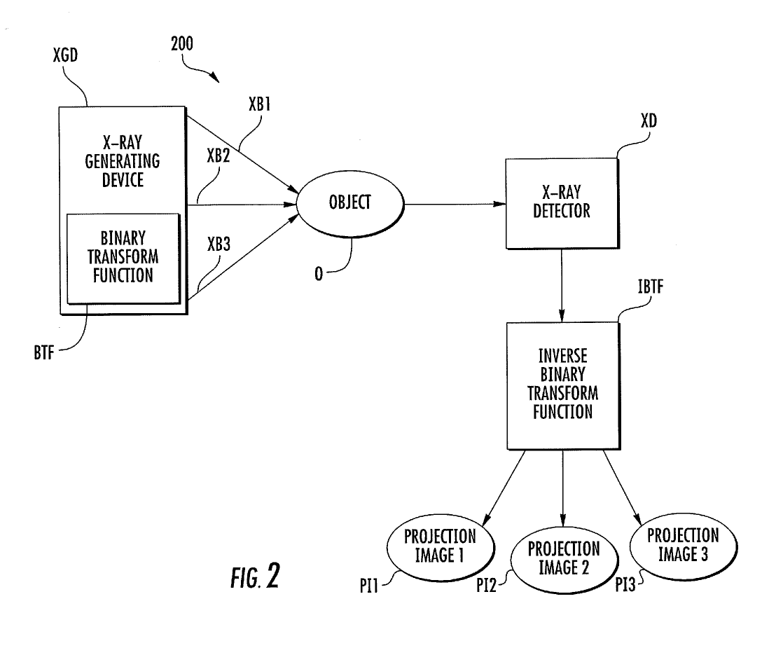

FIG. 2 is a block diagram of an exemplary BMXR system generally designated 200 for obtaining a multi-projection image of an object according to another aspect of the subject matter described herein. Referring to FIG. 2, binary transform function BTF can control multi-projection x-ray generating device XGD to generate multiple x-ray beams XB1, XB2, and XB3 under predetermined binary states for irradiating object O from different projection angles. Further, x-ray beams can be emitted simultaneously and/or in a predetermined spatial pattern.

The binary state of the signals of x-ray beams XB1, XB2, and XB3 can be based on a predetermined binary transform. Particularly, the signals can follow the form of a predetermined binary transform matrix. The binary state of the signals of the x-ray beams can be based on a pattern of 0 and 1 elements in the predetermined binary transform matrix. The following equation represents an exemplary 3×3 binary transform matrix suitable for a source projecting three x-rays beams.

$${w}_{3 \times 3} = ( \begin{matrix}{w}_{11} & {w}_{12} & {w}_{13} \\ {w}_{21} & {w}_{22} & {w}_{23} \\ {w}_{31} & {w}_{32} & {w}_{33}\end{matrix} ) \qquad ( 1 )$$

After passing through object O, x-ray beams XB can be detected by x-ray detector XD. X-ray detector XD can continuously capture the composite x-ray beams XB. In one example, x-ray detector XD can include an array or a matrix of x-ray photo diode detectors for detecting x-ray beams. In another example, x-ray detector XD can include an array or a matrix of photon counting x-ray detector elements for detecting x-ray beams. Further, in some aspects, x-ray detector XD can be configured to record x-ray signals at a fast frame rate.

After all or at least a portion of x-ray beams XB are collected and stored as x-ray signal data in a memory, an inverse binary transform function IBTF can apply an inverse binary transform to the stored x-ray signal data to recover the original individual projection images PI1, PI2, and PI3. In this manner, each individual x-ray source can be turned on multiple times during the imaging process. Thus, data acquisition speed can be greatly enhanced due to more efficient use of the x-ray source. In some embodiments, the more efficient use of the x-ray source can enhance the imaging speed for CT and tomosynthesis.

According to one aspect of the subject matter disclosed herein, a Hadamard multiplexing radiography approach is provided. The Hadamard transform is a particular example of a binary matrix transform that can be used in accordance with the present subject matter. As noted previously, any other suitable binary transform can be used in conjunction with the disclosed subject matter. Hadamard transform includes encoding signals using a spatial modulation technique, which is inherently based on square waves (on/off state of the signal source) rather than trigonometric functions. Hadamard transform instruments can for example include a signal source, an encoding Hadamard mask configured based on a corresponding Hadamard matrix, a detector, and a demultiplexing processor. The Hadamard transform technique superposes signals according to the Hadamard matrix. The original signals can be directly recovered from the recorded multiplexed signals by applying the inversed Hadamard transformation.

X-ray source XS can be any suitable device operable to generate an x-ray beam for imaging an object. An exemplary x-ray source can be a field emission x-ray source, such as those described in U.S. Pat. No. 6,553,096 to Zhou et al., filed Oct. 6, 2000 and issued Apr. 22, 2003; U.S. Pat. No. 6,850,595 to Zhou et al., file Dec. 4, 2002 and issued Feb. 1, 2005; and U.S. Pat. No. 6,876,724 to Zhou et al., filed Jan. 22, 2002 and issued Apr. 5, 2005, the disclosures of which are incorporated by reference herein. It is to be understood, however, that the systems and methods of x-ray imaging disclosed herein are not limited to any particular type or configuration of x-ray source. Rather, the present systems and methods can be implemented using any of a variety of x-ray sources capable of generating a pulsed x-ray beam.

In some embodiments, a multi-pixel (or multi-beam) x-ray source including multiple field emission x-ray sources (or pixels) and operable based on Hadamard multiplexing radiography techniques is provided. The multi-pixel x-ray source can include a multi-pixel field emission cathode with a linear array of gated electron emitting pixels. FIG. 3 is a schematic, cross-section side view of a multi-pixel field emission x-ray source generally designated 300 according to an aspect of the subject matter described herein. Referring to FIG. 3, x-ray source 300 can include a plurality of electron field emitters FE1-FE3 for emitting electrons. Electron field emitters FE1-FE3 can comprise one or more carbon nanotubes (CNT) and/or other suitable electron field emission materials. Further, electron field emitters FE1-FE3 can be attached to a surface of respective cathodes C1-C3, conductive or contact line, or other suitable conductive material. Although three, linearly-arranged electron field emitters are shown in this example, a multi-pixel x-ray source in accordance with the subject matter described herein can include any suitable number and arrangement of electron field emitters.

Electron field emitters FE1-FE3 can be controlled by a suitable controller C including metal-oxide-semiconductor field-effect transmitter (MOSFET) circuitry MC and binary transform function BTF. Controller C can control voltage sources to apply voltages between electron field emitters FE1-FE3 and gate electrodes GE1-GE3, respectively, to generate respective electric fields for extracting electron from electron field emitters FE1-FE3 to thereby produce respective electron beams EB1-EB3. In particular, controller C can individually operate a plurality of MOSFETs in MOSFET circuitry MC for individually controlling field emitters FE1-FE3 to emit electrons. The drains of the MOSFETs can be connected to a corresponding one of cathodes C1-C3 for controlling electron beam emission by respective emitters FE1-FE3. The MOSFETs can be turned on and off by the individual application of high signal (e.g., 5 V) and a low signal (e.g., 0 V), respectively, to the gates of MOSFETs. When a high signal is applied to the gate of a MOSFET, a drain-to-source channel of the transistor is turned on to apply a voltage difference between a respective cathode C1-C3 and a respective gate electrode GE1-GE3. A voltage difference exceeding a threshold can generate an electric field between a respective cathode C1-C3 and a respective gate electrode GE1-GE3 such that electrons are extracted from respective electron field emitters FE1-FE3. Conversely, when a low voltage (e.g., 0 V) is applied to the gate of a MOSFET, a corresponding drain-to-source channel is turned off such that the voltage at a respective electron field emitter FE1-FE3 is electrically floating and the voltage difference between a respective cathode C1-C3 and a respective gate electrode GE1-GE3 cannot generate an electric field of sufficient strength to extract electrons from the respective electron field emitter FE1-FE3. In one example, each x-ray pixel can provide a tube current of between 0.1 and 1 mA at 40 kVp. Controller C is operable to apply voltage pulses of different frequencies to the gates of the MOSFETs. Thus, controller C can individually control the frequencies of the electron beam pulses from field emitters FE1-FE3.

Further, in some embodiments x-ray source 300 can include an anode A having a plurality of focus spots bombarded by a corresponding electron beam. A voltage difference can be applied between anode A and gate electrodes GE1-GE3 such that respective fields are generated for accelerating electrons emitted by respective electron field emitters FE1-FE3 toward respective target structures of anode A. The target structures can produce x-ray beams having predetermined signals upon bombardment by electron beams EB1-EB3. X-ray source 300 can include focusing electrodes FEL1-FEL3 for focusing electrons extracted from respective electron field emitters FE1-FE3 on the target structures and thus reduce the size of electron beams EB1-EB3. Focusing electrodes FEL1-FEL3 can be controlled by application of voltage to focusing electrodes FEL1-FEL3 by a voltage source. The gate voltage can be varied depending on required flux. In one example, the focal spot size of each electron beam EB1-EB3 on anode A is about 200 μm.

Electron field emitters FE1-FE3 and gate electrode GE1-GE3 can be contained within a vacuum chamber with a sealed interior at about 10⁻⁷ torr pressure. The interior of the vacuum chamber can be evacuated to achieve a desired interior pressure. Electron beams EB1-EB3 can travel from the interior of the vacuum chamber to its exterior through an electron permeable portion or window. In one example, the electron permeable portion or window can be a 4″ diameter beryllium (Be) x-window. X-ray beams having distinct signals can be generated by the electron bombardment of anode A by electron beams of distinct signals. Further, anode A can be suitably shaped and/or angled such that the generated x-ray beams are transmitted toward an object from a plurality of different viewing angles.

In one aspect, binary transform function BTF can control MOSFET circuitry MC to turn off and on electron field emitters FE such that electron beams EB1-EB3 carry signals in a pattern of 0 and 1 elements in a predetermined Hadamard binary transform matrix. Corresponding x-ray beams generated by bombardment of anode A with electron beams EB1-EB3 can also carry the same signals in the pattern of 0 and 1 elements in the Hadamard binary transform matrix. Spatial modulation, or coding, of waveforms of the x-ray beam radiation generated by x-ray source 300 can be readily achieved through binary transform function BTF. The generated x-ray beams can be directed towards an object for irradiation with composite x-ray beams including signals based on the predetermined Hadamard binary transform. Anode A can be configured in a reflection mode for redirecting x-ray beams towards an object to be irradiated.

In one embodiment, an x-ray source including multi-beam pixels can include a field emission cathode with a linear array of gated carbon nanotube (CNT) emitting pixels, focusing electrodes, and a molybdenum target configured in a reflection mode. In some embodiments, these components can be housed in a vacuum chamber with a 4″ diameter Be x-ray window at a base pressure of 10⁻⁷ torr. Each emitting pixel can include a 1.5 mm diameter carbon nanotube film deposited on a metal surface, a 150 μm thick dielectric spacer, and an electron extraction gate made of a tungsten grid. Further, each emitting pixel can emit 1 mA current and can be evenly spaced with a center-to-center spacing of about 1.27 cm. The anode voltage can be set at 40 kV. Gate voltage can vary depending on the flux required. Switching the x-ray beam from each pixel can be controlled by sweeping a 0-5 Volt DC pulse through a corresponding MOSFET.

The carbon nanotube film can be deposited on the metal substrate by electrophoresis. The film can have a thickness of about 1.5 mm. The film can be coated on a metal disk. All of the gate electrodes can be electrically connected. An active electrostatic focusing electrode can be placed between the gate electrode and the anode for each pixel. The electron beam can be focused into a focus area on the anode target (referred to as a focal spot) when an electrical potential is applied onto the focusing electrode. Each emitting pixel can be connected to the drain of an n-channel MOSFET, the source of which is grounded. The gate of the MOSFET can be connected to the output of a digital I/O board, which can provide a 5 V DC voltage signal.

To generate x-ray radiation, a constant DC voltage can be applied to the anode and a variable DC voltage (less than about 1 kV) can be applied to the gate electrodes. MOSFET circuitry can be used to turn on and off the emission current from the individual pixels. To activate a pixel, a 5 V signal can be applied to open the channel of a corresponding MOSFET such that the pixel forms a complete electrical circuit with the gate electrode. Electrons can be emitted from the activated pixel when the gate voltage is larger than the critical field for emission. The electrons can be accelerated by the anode voltage and bombarded on a directly opposing area on the anode to produce x-ray radiation. Other, non-activated pixels will not emit electrons because they form an open circuit. To generate a scanning x-ray beam from different origins on the target, a pulsed controlling signal with predetermined pulse width can be swept across the individuals MOSFETs. At each point, the channel can be opened to generate an electron beam from the particular pixel which produced an x-ray beam from the corresponding focal point on the target.

A subset of pixels can be activated such that they all emit electrons with either the same or different pulsing frequencies which generate x-ray beams from different focal points with either the same or different frequencies. In one example, this can be accomplished by using separate gate electrodes for the field emission pixels. Extraction voltages can be applied to the corresponding pixels with the desired pulsing frequencies to generate field emitted electrons with the desired pulsing frequencies and amplitudes. In another example, a common gate can be used for all of the electron emitting pixels. Pulsing of the electron beam can be accomplished by pulsing the activation voltage applied to the MOSFET circuit. For example, to generate a pulsed x-ray with a desired frequency f, a pulsed voltage with the same frequency f can be applied to open the corresponding MOSFET.

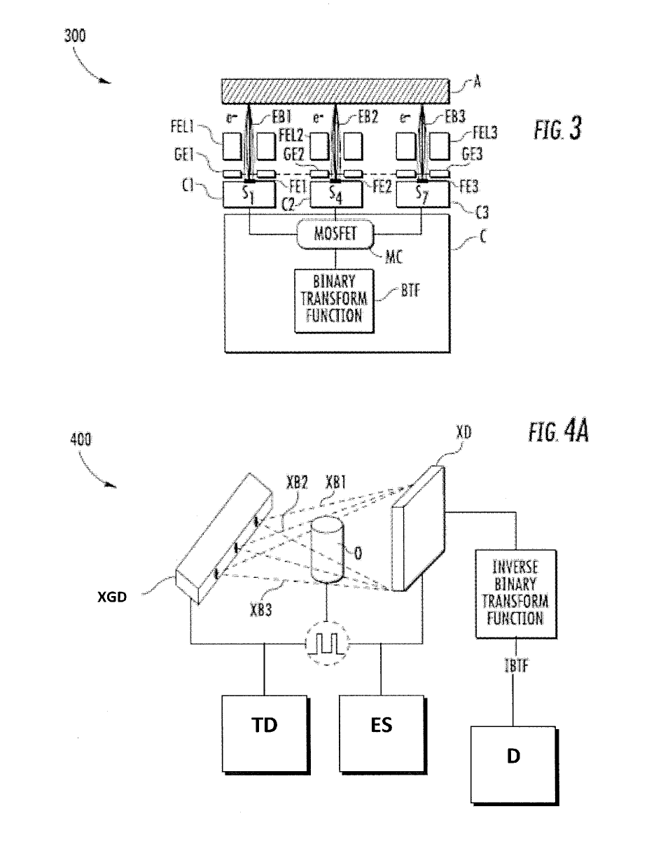

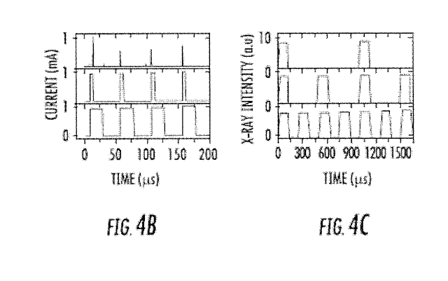

FIG. 4A is a perspective view of an x-ray imaging system generally designated 400 including x-ray generator device XGD for applying binary multiplexing x-ray radiography to object O according to an embodiment of the subject matter described herein. Referring to FIG. 4A, x-ray generator device XGD can include an x-ray source, such as x-ray source 300 shown in FIG. 3, for generating x-ray beams XB1-XB3 to irradiate object O. X-ray beams XB1-XB3 are shown in broken lines. Further, the beams are directed such that at least a portion of object O can be irradiated by each of the beams. X-ray beams XB1-XB3 can be generated by the bombardment of respective electron beams EB1-EB3 on anode A (shown in FIG. 3).

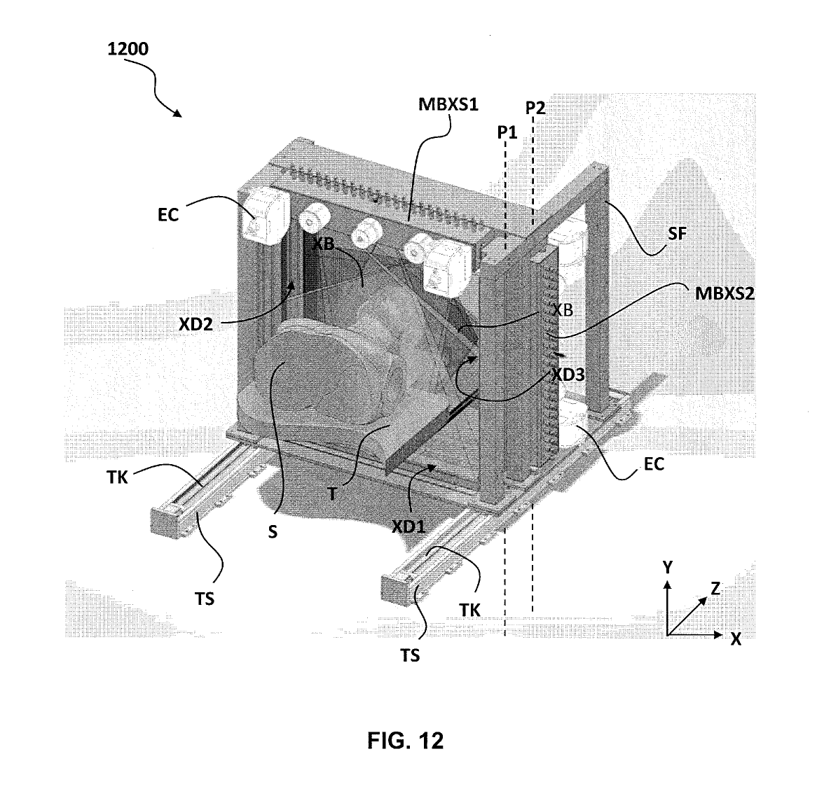

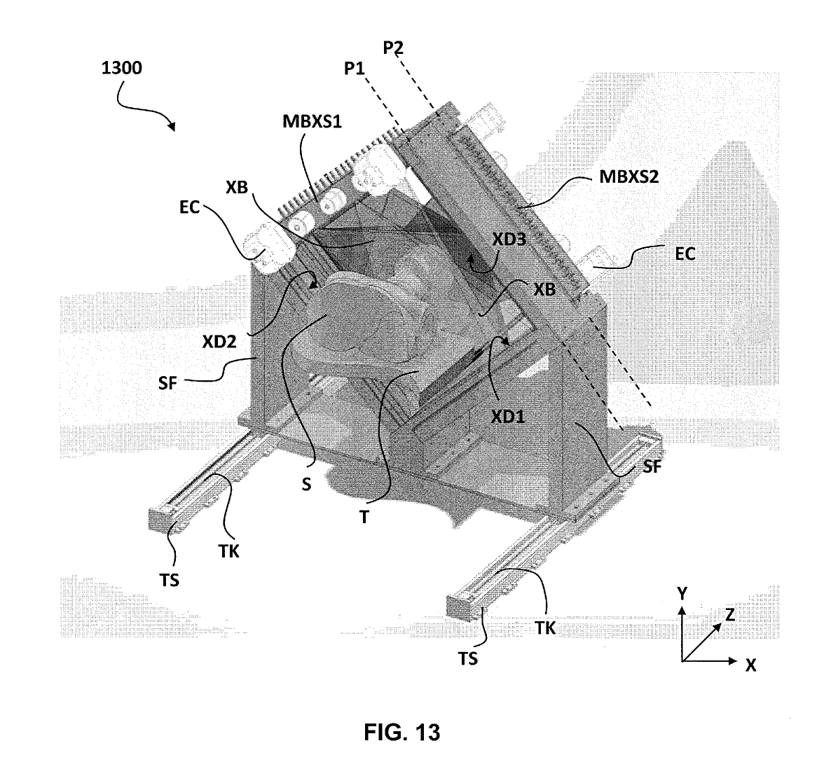

Object O can be placed on a sample stage (or Table T as depicted in FIGS. 12 and 13 depicted below) in position for intercepting x-ray beams XB1-XB3, which can carry signals in a pattern of 0 and 1 elements in a predetermined Hadamard binary transform matrix. The sample stage can be rotated for rotating of object O. The signal pattern of x-ray beams XB1-XB3 can correspond to the signal pattern contained in electron beams EB1 and EB3, which is based on the predetermined Hadamard binary transform matrix. All or a portion of x-ray beams XB1-XB3 can pass through object O.

After passing through object O, x-ray beams XB1-XB3 can be detected by x-ray detector XD. X-ray detector XD can continuously capture the composite x-ray beams XB1-XB3. After all or at least a portion of x-ray beams XB1-XB3 are collected and stored as x-ray signal data in a memory, inverse binary transform function IBTF can apply an inverse binary transform to the stored x-ray signal data to recover the signals of the composite x-ray beams. In one example, x-ray detector XD can deliver a 264×264 full frame with 200 micron pixels and 16 frames per second, which is suitable for many high speed x-ray imaging applications. A display unit D can organize the recovered signals for displaying images of object O based on the recovered signals.

The 0 and 1 x-ray beam signals can be generated by pulsing x-ray beams XB1-XB3. The pulsed x-ray radiation can include a programmable pulse width and repetition rate. FIGS. 4B and 4C are graphs illustrating an example of pulsed current applied to the x-ray pixels and the generated x-ray intensities over a period of time for generating pulsed x-ray radiation. Referring to FIG. 4B, the x-ray tube current is shown with a variable pulse width down to 0.5 μs at a constant repetition rate of 20 kHz. Referring to FIG. 4C, x-ray pulses of variable repetition rate are shown at a constant width of 150 μs acquired from a Si—PIN photodiode detector.

In Hadamard multiplexing, the multiplexed signals are generated from original signals weighted by 0 and 1. Assuming the original signals have the form X=[x₁ x₂ . . . x_{N-1} x_{N}]T, the multiplexed signals Y=[y₁ y₂ . . . y_{N-1} y_{N}]^{T} are in general related to the original signals by the linear transform Y=SX. For a Hadamard transform, the S-matrices consist of only 1s and 0s, which correspond to the on/off state of the signal source. The inverse of such a matrix is obtained by replacing the elements in the matrix by −1s and scaling by 2/(n+1).

As an example for the S matrix of order N=3, the convolution process can be expressed succinctly in the matrix notation by the following equation:

$$[ \begin{matrix}{y}_{1} \\ {y}_{2} \\ {y}_{3}\end{matrix} ] = [ \begin{matrix}1 & 1 & 0 \\ 1 & 0 & 1 \\ 0 & 1 & 1\end{matrix} ] [ \begin{matrix}{x}_{1} \\ {x}_{2} \\ {x}_{3}\end{matrix} ] = [ \begin{matrix}{x}_{1} + {x}_{2} \\ {x}_{1} + {x}_{3} \\ {x}_{2} + {x}_{3}\end{matrix} ] \qquad ( 2 )$$

The original signals can be recovered from the multiplexed signals by applying the inversed Hadamard matrix to both sides of equation (2), as illustrated by the following equation (3):

$$[ \begin{matrix}{x}_{1} \\ {x}_{2} \\ {x}_{3}\end{matrix} ] = \frac{2}{N ( = 3 ) + 1} [ \begin{matrix}1 & 1 & - 1 \\ 1 & - 1 & 1 \\ - 1 & 1 & 1\end{matrix} ] [ \begin{matrix}{y}_{1} \\ {y}_{2} \\ {y}_{3}\end{matrix} ] \qquad ( 3 )$$

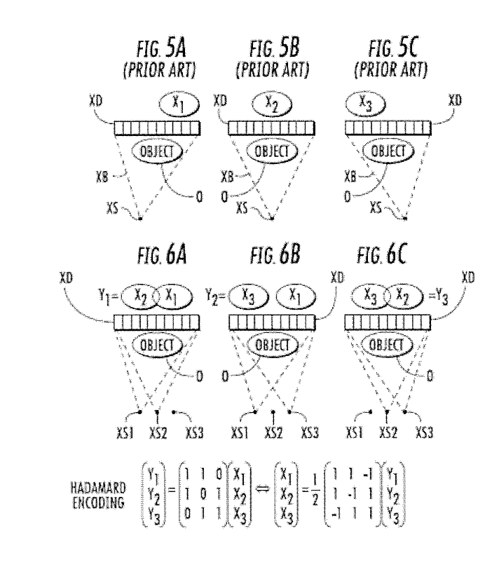

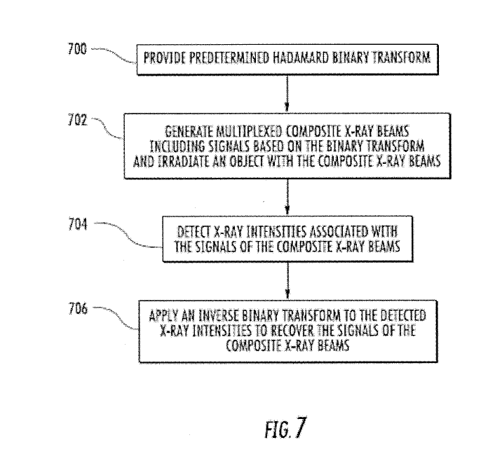

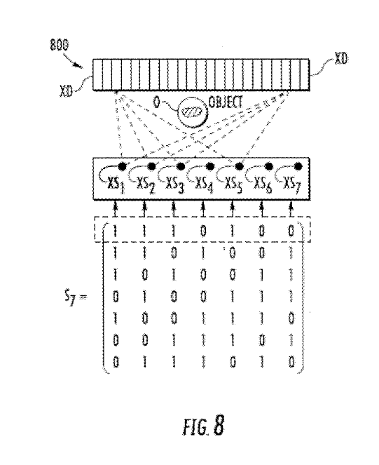

For Hadamard multiplexing radiography of order N=3, an exemplary comparison of conventional imaging and data processing procedures with procedures Hadamard techniques according to the subject matter described herein are described with respect to FIGS. 5A-5C and 6A-6C. FIGS. 5A-5C illustrate conventional scanning sequential imaging of object O by an x-ray detector XD and x-ray source XS at times t1-t3, respectively. Referring to FIGS. 5A-5C, projection images of object O are collected sequentially at times t1-t3 in the time domain by irradiating object O with x-ray beam XB. Object O is irradiated by x-ray beam XB from different projection angles by x-ray source XS. X-ray detector XD detects a portion of x-ray beam XB passing through object O. The total imaging time is 3Δt, assuming exposure time Δt for each projection image.

For the Hadamard multiplexing imaging example shown in FIGS. 6A-6C, the total number of exposures is 3, the same as in the FIGS. 5A-5C example. However, in the example of FIGS. 6A-6C, only two of x-ray sources XS1, XS2, and XS3 are turned on simultaneously for each exposure of object O to x-ray beams XB1, XB2, and XB3 according to the 1/0 (on/off) pattern of the Hadamard matrix shown in equation (2) above. Since each individual x-ray source XS1-XS3 is turned on twice during the three exposures to keep the dose constant, the exposure time for each frame can be reduced to Δt/2. The overall exposure time for the FIGS. 6A-6C example is 1.5Δt compared to 3Δt for sequential imaging as in the FIGS. 5A-5C example. In general, for Hadamard multiplexing radiography of order N, the data acquisition rate can be improved by a factor of (N+1)/2. Considering the fact that the order of 1,000 images (N˜1,000) are required for each gantry rotation in CT scanning, a large gain is provided in imaging speed in techniques according to the subject matter described herein, i.e. non-rotating stationary gantry scanning. On the other hand, since each x-ray source is turned on (N+1)/2 times during the entire data collection process, thus keeping the total exposure time fixed, the maximum workload of the x-ray tube needed to achieve the same x-ray dose for each image can be reduced by at least a factor of (N+1)/2.

FIG. 7 is a flow chart illustrating an exemplary process of binary multiplexing x-ray radiography according to an embodiment of the subject matter described herein. In this example, the process is based on a Hadamard binary transform, although any other suitable binary transform may be utilized. Referring to FIG. 7, a predetermined Hadamard binary transform can be provided (block 700). For example, the binary transform can be stored in a memory. The Hadamard binary transform can be based on a Hadamard matrix such as the matrix represented by the following equation (4):

$${S}_{N} = ( \begin{matrix}{s}_{11} & … & {s}_{1 N} \\ … & … & … \\ {s}_{N 1} & … & {s}_{N N}\end{matrix} ) \qquad ( 4 )$$

In this example, the multiplexing radiography is order N=7, although any other order may be used based on the number of x-ray sources or pixels.

In block 702, a binary transform function can generate multiplexed composite x-ray beams including signals based on the predetermined Hadamard binary transform and irradiate an object with the composite x-ray beams. For example, FIG. 8 is a schematic diagram illustrating an exemplary BMXR system generally designated 800 operable to generate multiplexed composite x-ray beams including signals based on the predetermined Hadamard binary transform and irradiate an object with the composite x-ray beams according to an embodiment of the subject matter described herein. Referring to FIG. 8, system 800 includes a plurality of x-ray sources (or pixels) XS1-XS7 configured to produce x-ray beams including signals based on a predetermined binary transform such as a Hadamard transform.

X-ray sources XS1-XS7 are individually addressable x-ray pixels. Each field emission pixel can comprise a gated carbon nanotube field emission cathode, a tungsten mesh extraction gate, and an electrostatic focusing lens. The cathode can be a random carbon nanotube composite film deposited on a metal substrate by electrophoresis. A MOSFET-based electronic circuit can control the on/off pattern of the x-ray sources.

The x-ray beams generated by x-ray sources XS1-XS7 can be controlled by a binary transform function to include signals based on the Hadamard binary transform matrix S₇. X-ray beams of x-ray sources XS1-XS7 can be applied to object O in sequence until the composite x-ray beams have been applied. Object O can be positioned on a sample stage. The first application of x-ray beam signals (shown in FIG. 8) include turning on x-ray sources XS1, XS2, XS3, and XS5 in accordance with the Hadamard binary transform matrix S₇.

As shown in the Hadamard binary transform matrix S₇ of FIG. 8, the 1s and 0s in each row of the Hadamard matrix are used to control the on (1) and off (0) state of a corresponding x-ray source XS1-XS7. To generate a multiplexing x-ray beam from multiple origins on object O, a controlling signal with a predetermined signal pattern and pulse width was swept across a control circuit (such as a MOSFET control circuit). A total number of N=7 multiplexed images can be acquired based on the following signal sequences: (1110100); (1101001); (1010011); (0100111); (1001110); (0011101); and (0111010). The seven multiplexed images can be collected and stored in a memory.

In the first application, x-ray sources XS4, XS6, and XS7 are turned off. The sequence of x-ray beams includes six more applications x-ray beam signals. The control of x-ray beam sources XS1-XS7 to apply the applications is shown in the Hadamard binary transform matrix S₇. The generation of each multiplexed image is based on the corresponding row of the Hadamard matrix. The on/off state of each x-ray source is determined by the 1/0 matrix element in that row.

Referring again to FIG. 7, x-ray intensities associated with the signals of the composite x-ray beams can be detected (block 704). For example, x-ray detector XD shown in FIG. 8 can detect the x-ray intensities associated with the signals of the composite x-ray beams generated by x-ray sources XS1-XS7. X-ray detector XD can be a single flat panel x-ray detector. The x-ray intensities comprise multiplexed x-ray images.

In block 706, an inverse binary transform can be applied to the detected x-ray intensities associated with the signals of the composite x-ray beams to recover the signals of the composite x-ray beams. For example, an inverse binary transform function can apply an inverse binary transform to the detected x-ray intensities associated with the signals of the composite x-ray beams to recover the signals of the composite x-ray beams. In one example, after a complete set of multiplexed images are collected, a demultiplexing algorithm based on a corresponding inversed Hadamard transform matrix can be applied to the complete set of multiplex images to recover the original projection images.

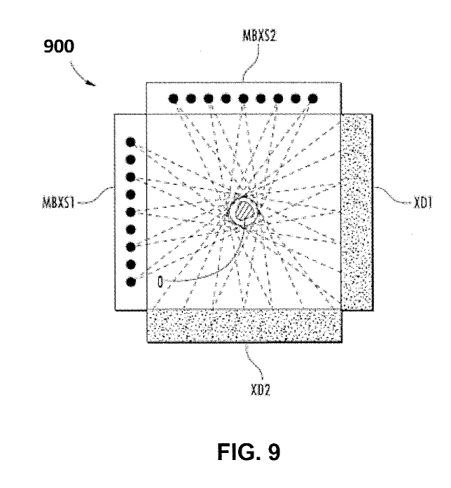

Systems and methods in accordance with the subject matter described herein can also be included in a CT imaging system having multi-beam field emission pixels. FIG. 9 is a schematic diagram of an exemplary CT imaging system generally designated 900 having multi-beam field emission pixels according to an embodiment of the subject matter described herein. Referring to FIG. 9, system 900 can comprise multi-beam x-ray sources MBXS1 and MBXS2 that each include multiple pixels configured for directing x-ray beams at object O. The pixels of x-ray sources MBXS1 and MBXS2 can be controlled to turn on and turn off in a signaling pattern based on a predetermined binary transform. Object O can be positioned for irradiation by the x-ray beams. X-ray detectors XD1 and XD2 can be area x-ray detectors configured to detect x-ray beams from x-ray sources MBXS1 and MBXS2, respectively. An inverse binary transform function can receive detected x-ray intensity data associated with the signals and apply an inverse binary transform to the detected x-ray intensities associated with the signals of the composite x-ray beams to recover the signals of the composite x-ray beams. Images can be generated based on the recovered signals.

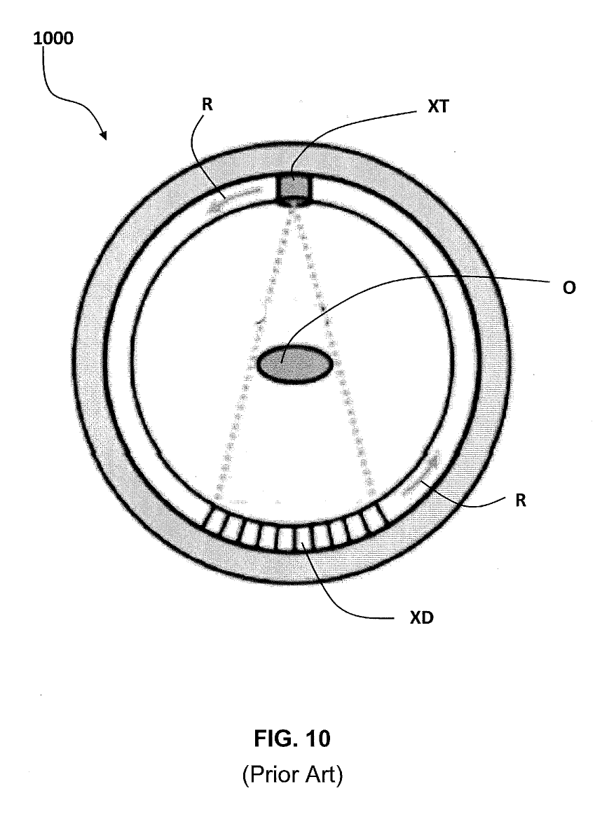

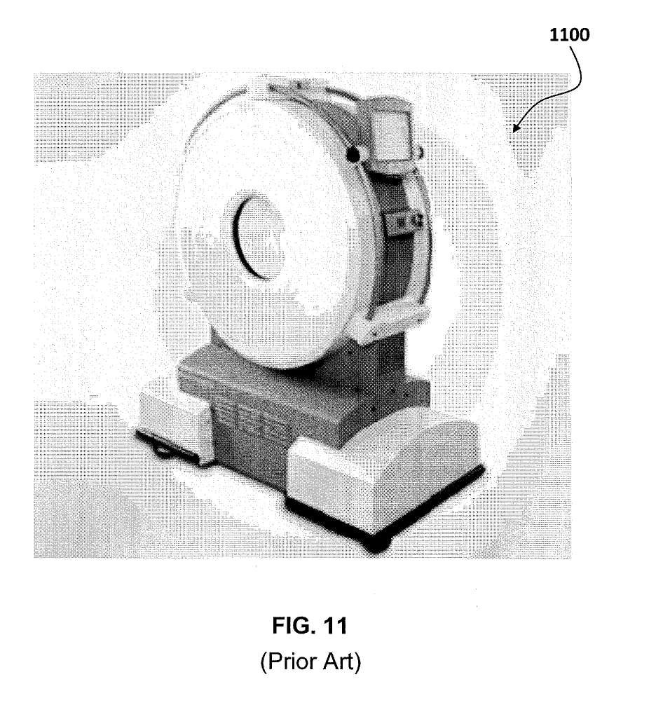

A CT scanner configured as depicted in FIG. 9 and disclosed herein can generate the projection views needed for CT reconstruction by electronically activating the individual x-ray sources (or x-ray “pixels”) without mechanically moving the x-ray source. That is, a CT scanner configured as depicted in FIG. 9 does not rotate around the subject like a traditional rotating gantry CT design. Such a traditional rotating gantry CT design is depicted in FIGS. 10 and 11. FIG. 10 is a schematic of a rotating gantry CT, generally depicted as 1000, comprising an x-ray tube XT arranged opposite an x-ray detector XD in a circular housing CH. In a rotating gantry CT scanner 1000 the object O to be scanned is placed in the circular housing CH and the x-ray tube XT and x-ray detector XD rotate around object O in a rotational direction R during the scanning procedure to thereby capture multiple images for CT reconstruction. An exemplary rotational gantry CT scanner 1100 is depicted in FIG. 11. As can be seen in FIG. 11, the portability and mobility of such a design is limited due to its size and mechanical configuration. By eliminating the rotational portion of the design the size and mechanical complexity can be minimized, and the portability and mobility can be greatly improved. Such advantages are provided by the non-rotating stationary gantry CT scanner disclosed herein.

A non-rotating stationary gantry CT scanner, generally referred to as 1200, is schematically depicted in FIG. 12. Computed tomography scanner 1200 can be configured to take a CT scan of an object or subject S, such as for example a human patient. More particularly, CT scanner 1200 can, without limitation, be used for imaging the head, spine and neck of subject S. Computed tomography scanner 1200 can comprise one or more multi-beam x-ray sources MBXS, one or more X-ray detectors XD, and a table T for holding an object or subject S to be scanned. The one or more multi-beam x-ray sources MBXS and the one or more X-ray detectors XD can be arranged in a support frame SF to form a polygon surrounding table T and subject S, e.g. a human patient, on table T such that a portion of subject S passing through the polygon configuration of CT 1200 can be imaged.

Computed tomography scanner 1200 can comprise one or more multi-beam x-ray sources, such as those discussed herein and depicted in FIGS. 3, 4A, 8 and 9, including for example a linear spatially distributed field emission x-ray source. In some embodiments, and as depicted in FIG. 12, CT scanner 1200 can comprise multi-beam x-ray sources MBXS1 and MBXS2 that each includes multiple pixels configured for directing x-ray beams XB at subject S. The pixels of x-ray sources MBXS1 and MBXS2 can be controlled to turn on and turn off in a signaling pattern based on a predetermined binary transform. In some embodiments, an x-ray source array can generate multiple x-ray beams from different projection angles, wherein the focal spots of the x-ray beams are arranged in a variety of geometrical configurations including linear array, circular array and 2-dimensional matrix with either regular or irregularly spacing. In some embodiments, the number of x-ray generating focus spots in the x-ray source array can be greater than 50 and less than 300.

Subject S can be positioned for irradiation by the x-ray beams XB (for illustrative purposes only two of the plurality of x-ray beams XB are labeled in FIG. 12). In some embodiments, CT scanner 1200 can comprise linear x-ray source arrays that can be used where the multiple x-ray focal spots are arranged in a straight line within each array. In some embodiments, each linear field emission x-ray source arrays can comprise between 20 and 300 focal spots arranged in a linear array inside the a vacuum envelope. In some embodiments, the field emission x-ray source array is adapted to uses carbon nanotube based materials as the field emission cathode. The corresponding x-ray detectors can be either line detectors or area detectors. In the case of line detectors, the array(s) can comprise either single row or multiple rows. In some embodiments, the energy of the x-ray source arrays can be rapidly switched between multiple x-ray energy levels for dual or multiple energy CT imaging.

In some embodiments, CT scanner 1200 can comprise an electronic control EC for controlling x-rays from x-ray sources MBXS1 and MBXS2, and particularly from individual x-ray focus spots. In some embodiments, electronic control EC can control a programmable photon flux and pulse sequence for synchronization of x-ray exposure with data collection of the one or more x-ray detector arrays. For example, in some embodiments x-ray sources MBXS1 and MBXS2 can be programmed so that the x-ray exposure can be synchronized with the detector readout and the motion of the object or subject S. To reduce the imaging blur from the cyclic motion, the x-ray exposure can be substantially shorter than the detector integration time. With this short exposure, the x-ray flux from the x-ray sources can be so limited that multiple frames are required for dynamic x-ray imaging of subject S in sequential imaging mode. In some embodiments, electronic control EC is configured to synchronize the x-ray pulse sequence with respiration or cardiac signals of a subject S being scanned to enable a perspective gated CT image of the subject.

In some embodiments, and as depicted in FIG. 12, MBXS1 can be configured in a substantially horizontal position such that it is parallel to plane X, while MBXS2 is configured in a substantially vertical position such that it is parallel to plane Y. By configuring MBXS1 and MBXS2 to lie in planes X and Y, x-ray beams XB emitted from one or more pixels of MBXS1 and MBXS2 can cover substantially all angles around the subject S being imaged. Since the arrangement in FIG. 12 is for illustrative purposes only, it can be appreciated that a CT scanner as depicted in FIG. 12 can comprise 1, 2, 3, 4, 5, 6, 7, 8, 9, 10 or more multi-beam x-ray sources without departing from the scope of the instant disclosure.

In some embodiments, x-ray detectors XD can be area x-ray detectors configured to detect x-ray beams from x-ray sources MBXS, such as for example MBXS1 and MBXS2. In some embodiments, one or more x-ray detector XD arrays can be configured to detect x-ray energy from one or more energy bins. In some embodiments, CT scanner 1200 can be configured to have one x-ray detector XD that corresponds to, and is arranged adjacent to and across from, or in a position opposing, each x-ray source MBXS. For example, in FIG. 12, x-ray detector XD1 can be arranged or positioned directly opposite x-ray source MBXS1 to thereby detect x-rays emitted from x-ray source MBXS1. Such a configuration can be replicated for each x-ray sources MBXS in a given CT scanner 1200.

Alternatively, in some embodiments CT scanner 1200 can comprise a plurality of x-ray detectors XD for each x-ray source MBXS. As depicted in FIG. 12, x-ray detectors XD1, XD2, and XD3 are arranged on three separate sides of a square configuration with x-ray source MBXS1 arranged on the fourth side. Such a configuration forms a square structure in a first imaging plane P1. X-ray detectors XD1, XD2, and XD3 can detect x-rays emitted from x-ray source MBXS1 and record images of subject S within first imaging plane P1. Correspondingly, x-ray source MBXS2 can be arranged with three x-ray detectors in a similar manner thereby forming a similar square structure in a second imaging plane P2. As depicted in FIG. 12, second imaging plane P2 is behind and parallel to first imaging plane P1. As such, the three x-ray detectors paired with x-ray source MBXS2 are not visible in FIG. 12. As depicted in FIG. 12, x-ray source MBXS2 and its corresponding x-ray detectors forming a square configuration in second imaging plane P2 are rotated 90 degrees on the Z axis with respect to x-ray source MBXS1 in first imaging plane P1. Such an arrangement provides for x-ray beams XB from x-ray sources MBXS1 and MBXS2 to originate from different angles to thereby provide for images to be captured from a greater number of angles. Thus, as depicted in FIG. 12, six different x-ray detectors XD are provided (XD1, XD2, and XD3 in first imaging plane P1, corresponding to x-ray source MBXS1, and three x-ray detectors (not visible) in second imaging plane P2, corresponding to x-ray source MBXS2).

In some embodiments, the one or more x-ray detector arrays detect only the x-ray radiation from the one or more x-ray source arrays in the same imaging plane. Such a configuration can be replicated for additional x-ray sources and corresponding x-ray detectors in additional planes layered behind first and second imaging planes P1 and P2 without departing from the scope of the instant disclosure. For example, three x-ray source arrays and corresponding detector arrays can be arranged in a substantially parallel configuration. In some embodiments, three or more x-ray source arrays and corresponding detector arrays can be arranged in a substantially parallel configuration to form three or more parallel imaging planes. In some embodiments, the orientations of the x-ray source arrays and detector arrays in each imaging plane are off-set from each other to provide increased angular coverage. In some embodiments, the x-ray source arrays in parallel imaging planes are offset by 90 degrees with respect one another. Moreover, for each x-ray source in a given imaging plane any number of x-ray detectors can be paired therewith to detect the emitted x-rays. For example, while the configuration in FIG. 12 is a square configuration, the x-ray sources can be paired with a plurality of x-ray detectors in any polygon configuration that permits a subject S to be scanned. In some embodiments, one or more x-ray detector arrays can be arranged in a linear, L-shape, U-shape, or irregular polygon shape that is opposite to the linear x-ray source array.

In some embodiments, CT scanner 1200 can further comprise one or more translation stages TS, as depicted in FIG. 12. Translation stages TS can comprise a track TK system to allow for CT scanner 1200 to slide or move along the Z axis to thereby scan a section or portion of subject S. In some embodiments, this lateral translation along the Z axis is automatic or manual. Alternatively, or in addition, table T can be configured to move along the Z axis to facilitate the insertion of subject S into the polygon of CT scanner 1200 where the scanning takes place, and/or to facilitate scanning of subject S, or a portion thereof, alone the Z axis. Scanner 1200, and particularly support frame SF, can be mechanically coupled to one or more translation stages TS using any suitable means, such as rollers, bearings, slides, etc., to facilitate movement along the Z axis.

When in use, a CT scanner 1200 as depicted in FIG. 12, can provide for two imaging planes (P1 and P2). For each imaging plane, the x-ray source, such as MBXS1 or MBXS2, generates x-rays from different viewing angles by electronically scanning the individual x-ray sources. The projection images are captured by the detector arrays, such as XD1, XD2 and/or XD3, which can be arranged in a U-shape (or on three sides of the square geometry) and can be positioned in the same plane as the x-ray source array. The second imaging plane (P2) can be parallel to the first one (P1), but the x-ray array source can be rotated by 90 degrees to cover more imaging angles. No system rotation is performed during imaging acquisition. The system can be equipped with translation stages TS to allow for imaging along the Z axis. The subject S, or patient, can lie on a metal-free bed, stretcher, or table T, which in some embodiments can comprise a head holder, which reduces the artifacts during imaging. In some embodiments the system can move along the Z axis to perform a full scan of the head, neck and/or spine of subject S.

FIG. 13 depicts another embodiment of a non-rotating stationary gantry CT scanner, generally depicted as 1300. Computed tomography scanner 1300 shares substantially the same components as CT scanner 1200 depicted in FIG. 12, but in a different configuration. In particular, CT scanner 1300 comprises a square geometry that is rotated by 45 degrees as compared to the square geometry of CT scanner 1200. The orientation of CT scanner 1300 provides a wider opening for subject S to enter the scanner. In order to facilitate the orientation depicted in FIG. 13, support frame SF of CT scanner 1300 differs from that of CT scanner 1200. Otherwise, the components of CT scanner 1200 and CT scanner 1300, including the nomenclature used in FIGS. 12 and 13, can be at least substantially the same.

In particular, CT scanner 1300 can be configured to take a CT scan of an object or subject S, such as for example a human patient. More particularly, CT scanner 1300 can be used for imaging the head, spine and neck of subject S. Computed tomography scanner 1300 can comprise one or more multi-beam x-ray sources MBXS, one or more X-ray detectors XD, and a table T for holding an object or subject S to be scanned. The one or more multi-beam x-ray sources MBXS and the one or more X-ray detectors XD can be arranged in a support frame SF to form a polygon surrounding table T and subject S, e.g. a human patient, on table T such that a portion of subject S passing through the polygon configuration of CT 1300 can be imaged.

Computed tomography scanner 1300 can comprise one or more multi-beam x-ray sources, such as those discussed herein and depicted in FIGS. 3, 4A, 8 and 9, including for example a linear spatially distributed field emission x-ray source. In some embodiments, and as depicted in FIG. 13, CT scanner 1300 can comprise multi-beam x-ray sources MBXS1 and MBXS2 that each includes multiple pixels configured for directing x-ray beams XB at subject S. The pixels of x-ray sources MBXS1 and MBXS2 can be controlled to turn on and turn off in a signaling pattern based on a predetermined binary transform. Subject S can be positioned for irradiation by the x-ray beams XB (for illustrative purposes only two of the plurality of x-ray beams XB are labeled in FIG. 13). In some embodiments, CT scanner 1300 can comprise linear x-ray source arrays that can be used where the multiple x-ray focal spots are arranged in a straight line within each array. In some embodiments, each linear field emission x-ray source arrays can comprise between 20 and 300 focal spots that arranged in a linear array inside the a vacuum envelope. In some embodiments, the field emission x-ray source array is adapted to use carbon nanotube based materials as the field emission cathode. The corresponding x-ray detectors can be either line detectors or area detectors. In the case of line detectors, the array(s) can comprise either single row or multiple rows. In some embodiments, the energy of the x-ray source arrays can be rapidly switched between multiple x-ray energy levels for dual or multiple energy CT imaging.

In some embodiments, CT scanner 1300 can comprise an electronic control EC for controlling x-rays from x-ray sources MBXS1 and MBXS2, and particularly from individual x-ray focus spots. In some embodiments, electronic control EC can control a programmable photon flux and pulse sequence for synchronization of x-ray exposure with data collection of the one or more x-ray detector arrays. For example, in some embodiments x-ray sources MBXS1 and MBXS2 can be programmed so that the x-ray exposure can be synchronized with the detector readout and the motion of the object or subject S. To reduce the imaging blur from the cyclic motion, the x-ray exposure can be substantially shorter than the detector integration time. With this short exposure, the x-ray flux from the x-ray sources can be so limited that multiple frames are required for dynamic x-ray imaging of subject S in sequential imaging mode.

In some embodiments, and as depicted in FIG. 13, MBXS1 can be configured at a substantially 45 degree angle, while MBXS2 is also configured at a substantially 45 degree angle, such that the two are substantially perpendicular to one another. By configuring MBXS1 and MBXS2 at a 90 degree angle with respect to one another, the x-ray beams XB emitted from one or more pixels of MBXS1 and MBXS2 can cover substantially all angles around the subject S being imaged. Since the arrangement in FIG. 13 is for illustrative purposes only, it can be appreciated that a CT scanner as depicted in FIG. 13 can comprise 1, 2, 3, 4, 5, 6, 7, 8, 9, 10 or more multi-beam x-ray sources without departing from the scope of the instant disclosure.

In some embodiments, x-ray detectors XD can be area x-ray detectors configured to detect x-ray beams from x-ray sources MBXS, such as for example MBXS1 and MBXS2. In some embodiments, CT scanner 1300 can be configured to have one x-ray detector XD that corresponds to, and is arranged adjacent to and across from, or in a position opposing, each x-ray source MBXS. For example, in FIG. 13, x-ray detector XD1 can be arranged or positioned directly opposite x-ray source MBXS1 to thereby detect x-rays emitted from x-ray source MBXS1. Such a configuration can be replicated for each x-ray sources MBXS in a given CT scanner 1300.

Alternatively, in some embodiments CT scanner 1300 can comprise a plurality of x-ray detectors XD for each x-ray source MBXS. As depicted in FIG. 13, x-ray detectors XD1, XD2, and XD3 are arranged on three separate sides of a square configuration with x-ray source MBXS1 arranged on the fourth side. Such a configuration forms a square structure or geometry in a first imaging plane P1. X-ray detectors XD1, XD2, and XD3 can detect x-rays emitted from x-ray source MBXS1 and record images of subject S within first imaging plane P1. Correspondingly, x-ray source MBXS2 can be arranged with three x-ray detectors in a similar manner thereby forming a similar square structure in a second imaging plane P2. As depicted in FIG. 13, second imaging plane P2 is behind first imaging plane P1. As such, the three x-ray detectors paired with x-ray source MBXS2 are not visible in FIG. 13. As depicted in FIG. 13, x-ray source MBXS2 and its corresponding x-ray detectors forming a square configuration in second imaging plane P2 are rotated 90 degrees on the Z axis with respect to x-ray source MBXS1 in first imaging plane P1. Such an arrangement provides for x-ray beams XB from x-ray sources MBXS1 and MBXS2 to originate from different angles to thereby provide for images to be captured from a greater number of angles. Thus, as depicted in FIG. 13, six different x-ray detectors XD are provided (XD1, XD2, and XD3 in first imaging plane P1, corresponding to x-ray source MBXS1, and three x-ray detectors (not visible) in second imaging plane P2, corresponding to x-ray source MBXS2).

Such a configuration can be replicated for additional x-ray sources and corresponding x-ray detectors in additional imaging planes layered behind first and second imaging planes P1 and P2 without departing from the scope of the disclosure herein. In some embodiments, three or more x-ray source arrays and corresponding detector arrays can be arranged in a substantially parallel configuration to form three or more parallel imaging planes. In some embodiments, the orientations of the x-ray source arrays and detector arrays in each imaging plane are off-set from each other to provide increased angular coverage. In some embodiments, the x-ray source arrays in parallel imaging planes are offset by 90 degrees with respect one another. Moreover, for each x-ray source in a given plane any number of x-ray detectors can be paired therewith to detect the emitted x-rays. For example, while the configuration in FIG. 13 is a square configuration, the x-ray sources can be paired with a plurality of x-ray detectors in any polygon configuration that permits a subject S to be scanned.

In some embodiments, CT scanner 1300 can further comprise one or more translation stages TS, as depicted in FIG. 13. Translation stages TS can comprise a track TK system to allow for CT scanner 1300 to slide or move along the Z axis to thereby scan a section or portion of subject S. Alternatively, or in addition, table T can be configured to move along the Z axis to facilitate the insertion of subject S into the polygon of CT scanner 1300 where the scanning takes place, and/or to facilitate scanning of subject S, or a portion thereof, alone the Z axis. Scanner 1300, and particularly support frame SF, can be mechanically coupled to one or more translation stages TS using any suitable means, such as rollers, bearings, slides, etc., to facilitate movement along the Z axis.

When in use, a CT scanner 1300 as depicted in FIG. 13, can provide for two imaging planes (P1 and P2). For each imaging plane, the x-ray source, such as MBXS1 or MBXS2, generates x-rays from different viewing angles by electronically scanning the individual x-ray sources. The projection images are captured by the detector arrays, such as XD1, XD2 and/or XD3, which can be arranged in a U-shape (or on three sides of the square geometry) and can be positioned in the same imaging plane as the x-ray source array. The second imaging plane (P2) can be parallel to the first one (P1), but the x-ray array source can be rotated by 90 degrees to cover more imaging angles. No system rotation is performed during imaging acquisition. The system can be equipped with translation stages TS to allow for imaging along the Z axis: The subject S, or patient, can lie on a metal-free bed, stretcher, or table T, which in some embodiments can comprise a head holder, which reduces the artifacts during imaging. In some embodiments the system can move along the Z axis to perform a full scan of the head, neck and/or spine of subject S.

In some embodiments, a CT scanner 1200 or 1300 can be linked, either directly or wirelessly, to a central processing unit configured to collect, store, analyze and/or configure data and/or images collected from a scan of a subject or object. A central processing unit can comprise a processor and computer readable medium having stored thereon executable instructions that when executed by the processor control the central processing unit to perform such collection, storing, analyzing and/or configuring of data and/or images. In some embodiments, CT scanner 1200 or 1300 can comprise an image processing and display component to process projection images to form a three-dimensional reconstructed image of an object. In some embodiments, the imaging processing and display component is configured to process projection images to form a three-dimensional reconstructed image of an object and to display it in an image display apparatus.

In some embodiments, CT scanner 1200 or 1300 can be equipped with an electrical power generator, or other energy source (ES in FIG. 4A), such as for example a rechargeable battery.

In some embodiments, CT scanner 1200 or 1300, and/or the central processing unit, can be linked, either directly or wirelessly, to a display unit for viewing the scans, images and other output data. In some embodiments, CT scanner 1200 or 1300 can be equipped with wireless communication capability, or other transmission device (TD in FIG. 4A), for transmitting images and/or data to one or more centralized or desired location(s), such as a location where a radiologist or other medical professional can analyze the results.

In some embodiments, a central processing unit associated with a CT scanner 1200 or 1300 can comprise an inverse binary transform function. An inverse binary transform function can receive detected x-ray intensity data associated with the signals detected in a CT scanner 1200 or 1300 and apply an inverse binary transform to the detected x-ray intensities associated with the signals of the composite x-ray beams to recover the signals of the composite x-ray beams. Images can be generated based on the recovered signals.

In some embodiments, an advanced reconstruction algorithm such as iterative reconstruction methodology can be used. Iterative reconstruction techniques can be useful for the reconstruction of sparse CT data sets. That is, use of such a methodology can compensate for an incomplete data set in situations where not all viewing angles are covered. An advantage of such a technique is less projection views are needed. This can also result in reduction of the total imaging dose received by a patient. Though much of the work with iterative reconstruction techniques has been focused on dose reduction, the same principles can be applied to a solid state CT. Moreover, as few as approximately 60 projections, for example, are sufficient to reconstruct a simple head phantom, using a iterative reconstruction techniques comprising an algorithm called adaptive steepest descent-projection onto convex sets. In contrast, traditional Feldkamp based reconstruction techniques (used currently in almost all clinical CT scanners) require 180 projections or more to reconstruct a standard head CT. Other more classic iterative reconstruction techniques also require substantially more projections to appropriately reconstruct the image of interest. By taking advantage of the advanced iterative reconstruction techniques, the design of a solid-state (non-rotational stationary gantry) CT system as disclosed herein can be simplified through a significant reduction in the number of x-ray sources.

A CT scanner 1200 or 1300 can be utilized for computed tomography scans of objects and subjects, including human patients, to address indications and diagnostic needs as are currently the basis for traditional CT scans. In some embodiments, CT scanners 1200 and/or 1300 are configured to scan the head, spin and/or neck of a human subject believed to be suffering a hemorrhagic stroke. Since CT scanners 1200 and/or 1300 are non-rotational gantry designs, they are more portable and mobile and can therefore be taken to a patient in need. As such, on-site exclusion of hemorrhage could be performed to reduce the time to thrombolytic treatment in stroke victims, and increase the number of patients that can be treated during the critical early stages of stroke, including the currently understood “three hour golden window”. Identification of brain hemorrhage using a mobile CT scanner, such as CT scanners 1200 and/or 1300, in a mobile unit, such as in an ambulance, military vehicle or other mobile transport unit, could also allow earlier transportation to a tertiary care center, which has been shown to improve outcomes.

In some embodiments, a mobile CT scanner, such as CT scanners 1200 and/or 1300, could be used to evaluate a battle-field trauma in a subject to identify and/or diagnose a head injury. In this case mechanical robustness of the scanner is important. A stationary, or non-rotating-gentry, CT scanner has fewer mechanically moving parts as compared to traditional rotating-gentry CT scanners, and therefore will require less maintenance and will be less likely to be damaged from the harsh operating environment in some of the mobile military medical units. Portability is also desired for military medical units. For both ambulance and battlefield medical applications, the capability to wirelessly transmit data to a central facility where radiologists are on site to analyze the results is desired.

In some embodiments, methods of operating the disclose CT imaging systems are provided. Such methods can comprise configuring a stationary distributed x-ray source array to emit x-ray from one or more individually addressable focus spots, positioning an object to be imaged between the distributed x-ray source array and at least one x-ray detector, providing an electronic control unit to control the sequence and x-ray parameter of one or more individual x-ray beams from the x-ray source array, detecting the x-ray emitted from the x-ray source array, generating a visualization of one or more images of the object based on x-rays detected by the at least one x-ray detector, processing the one or more images to form a 3-dimensional reconstructed image of the object, and generating a visualization of the image in 2D or 3D in a display unit. In some embodiments, the electronic control unit synchronizes image acquisition with physiological signals of the object.

In some embodiments, a method of operating a CT imaging system can comprise configuring a stationary distributed x-ray source array to emit x-ray from one or more individually addressable focus spots, positioning an object to be imaged between the distributed x-ray source array and at least one x-ray detector, providing an electronic control unit to control the sequence and x-ray parameter of one or more individual x-ray beams from the x-ray source array, detecting the x-ray emitted from the x-ray source array, generating a visualization of one or more images of the object based on x-rays detected by the at least one x-ray detector, analyzing the generated visualization, and altering one or more image acquisition parameters based on the analysis of the generated visualization to generate an improved image. In some embodiments, the one or more image acquisition parameters to be changed is selected from the group consisting of: the number of projection views, the distribution of the project views, the keV and mAs from individual x-ray focus spots, the focus spot size, and the filtration of x-ray spectrum.

### EXAMPLE

The following example has been included to illustrate modes of the presently claimed subject matter. Certain aspects of the following example are described in terms of techniques and procedures found to work well in the practice of the presently claimed subject matter. This example illustrates standard laboratory practices of the co-inventors. In light of the present disclosure and the general level of skill in the art, those of skill will appreciate that the following example is intended to be exemplary only and that numerous changes, modifications, and alterations can be employed without departing from the scope of the presently claimed subject matter.

### Example 1

Preliminary data was acquired to demonstrate system feasibility with an existing CNT based linear x-ray source array and a low-resolution industrial linear detector. Z-axis coverage can then be increased by adding multiple linear detectors with resulting linearly scaled increase in speed, or by physical linear translation of the source detector combination. The translation over the axial field of view (FOV) of the head (approximately 23 cm) can be performed relatively inexpensively with high precision and speed with conventional mechanical components such as linear translation stages. Additional detectors could be added and used if there was a need for a significant increase in speed, such as for CT perfusion applications.

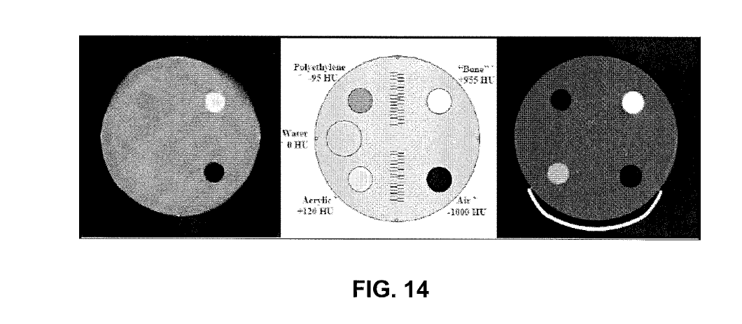

An image from the physical simulation of a potential geometry is illustrated in FIG. 14. Multiple passes from an existing 49 source linear array (spaced at 4 mm centers) were performed at 140 keV and 4.0 mAs exposure per slice. The scan configuration was based on a rectangle with a 62.9 cm edge-length. A XDAS-V3 linear x-ray detector (Sens-Tech, Berkshire UK) was utilized with pixel pitch of 1.6 mm and placed 62.9 cm away from the source. An American College of Radiology (ACR) CT accreditation phantom (Gammex 464) was scanned through stepped translation. After each pass, the linear source array and linear detectors were moved to new positions to complete the simulation of the potential geometry. The projections were then reconstructed using an iterative reconstruction technique, Total-variation minimization method with Algebraic Reconstruction Technique constraint (TV-ART).

The phantom images were then evaluated using the ACR standards. The mockup was not anticipated to achieve the ACR standards, but to serve as a basis for initial comparison. Four quality parameters are normally evaluated with the ACR phantom; Hounsfield unit reproduction, contrast to noise ratio and low contrast detectability, resolution and homogeneity.

Dose from the physical simulation was measured using a CTDI phantom and Radcal Accu-Pro Model 9096 (Radcal Corp, Monrovia, Calif., United States of America) under the simulated imaging conditions. A dose measurement was also obtained from a clinical Ceretom (Neurologica Corp, Danvers, Mass., United States of America) head only scanner with a 7 mA, 2 second exposure clinical head imaging protocol to serve as a basis for comparison.

A sample reconstructed slice of the ACR phantom is shown in FIG. 14 with a diagram corresponding to the makeup of the phantom at that slice. All four inserts (polyethylene, acrylic, air and bone) are visible in the reconstructed slice. The low contrast visibility component and resolution phantoms were non-diagnostic, the low contrast objects could not be observed, and the lowest resolution targets could not be distinguished (4 line pairs per mm). Contrast to noise of acrylic to water measured 7.3 (vs. 23.0 for the Ceretom) normalized to a clinical 5.0 mm slice. Dose measured 12.16 mrem for a single slice (vs. 592.45 mrem for the entire phantom measured on the Ceretom). The 4, 5 and 6 line pairs per cm target were readily identified using the Ceretom CT. Linearity of the system within a clinical range of Hounsfield units was estimated by extrapolating the value of the acrylic insert based on assumed air and water values. A value of 126.6 HU was determined, with reasonable agreement to the anticipated value (120 HU).

Given the lower sensitivity, detector resolution and dose of the physical simulation of a potential geometry, it was anticipated that image quality would be sufficiently less than the conventional head CT system. This is also demonstrated by the dose measurements, where the CNT system was approximately 50 times less than the conventional system. Extrapolating the expected CNR of the system at a conventional dose, it is anticipate that there would be a 7-fold increase, resulting in a CNR of approximately 49, over twice of the conventional system. Thus, based on this physical simulation, it is anticipated that a CT system as disclosed herein can readily obtain the image quality necessary to perform CT examinations at an appropriately low dose.

The present subject matter can be embodied in other forms without departure from the spirit and essential characteristics thereof. The embodiments described therefore are to be considered in all respects as illustrative and not restrictive. Although the present subject matter has been described in terms of certain preferred embodiments, other embodiments that are apparent to those of ordinary skill in the art are also within the scope of the present subject matter.

What is claimed is:

1. A stationary gantry x-ray computed tomography imaging system, comprising two imaging planes, the system comprising:
a first imaging plane comprising a first linear spatially distributed field emission x-ray source array for emitting x-ray radiation and a first x-ray detector array positioned opposing the first x-ray source array;
a second imaging plane comprising second linear spatially distributed field emission x-ray source array for emitting x-ray radiation and a second x-ray detector array positioned opposing the second x-ray source array, the two imaging planes being substantially parallel to each other, and the first and second x-ray source arrays being rotated by 90 degrees with respect to one another within the first and second imaging planes, respectively;
an electronic control for controlling x-rays from individual x-ray focus spots of the spatially distributed x-ray source array with programmable photon flux and pulse sequence for synchronization of x-ray exposure with data collection of the first and second x-ray detector arrays; and
the system being adapted to process and reconstruct collected images to form a three-dimensional reconstructed image of an object and to display the image in an image display apparatus.

2. The system of claim 1, wherein the system is adapted to use an iterative image algorithm for reconstruction.

3. The system of claim 1, wherein each x-ray source array comprises between 20 and 300 focal spots arranged in a linear array inside a vacuum envelope.

4. The system of claim 1, wherein the x-ray source array is adapted to use carbon nanotube based materials as the field emission cathode.

5. The system of claim 1, further comprising an electrical power generator, wherein the electrical power generator is a rechargeable battery.

6. The system of claim 1, further comprising a wired or a wireless device for transmission of the acquired images or reconstructed images to a remote interpretation station.

7. The system of claim 1, wherein the electronic control unit is adapted to synchronize x-ray pulse sequence with respiration or cardiac signals of a subject to enable a prospective gated computed tomography image of the subject.

8. The system of claim 7 wherein an electron field emission extraction voltage of the x-ray source array is synchronized with the respiration or cardiac signals.

9. The system of claim 1, wherein the imaging system is compact and portable such that it is usable inside patient transport vehicles in the field, or is mobile such that the system is movable from room to room.

10. The system of claim 1, wherein the entire system is adapted to automatically be translated along an axial axis to obtain a computed tomography image of a subject over a large field of view.

11. The system of claim 1, wherein the first and second detector arrays are adapted for detecting x-ray energy from one or more energy bins.

12. The system of claim 1, wherein energy of the first and second x-ray source arrays can be rapidly switched between multiple x-ray energy levels for dual or multiple energy CT imaging.

13. A stationary gantry x-ray computed tomography imaging system, comprising three or more parallel imaging planes, the system comprising:
three or more imaging planes wherein each imaging plane comprises a linear spatially distributed field emission x-ray source array for emitting x-ray radiation and an x-ray detector array positioned opposing the x-ray source array, wherein the orientation of the x-ray source arrays and detector arrays in each imaging plane are off-set from each other to provide increased angular coverage;
an electronic control for controlling x-rays from individual x-ray focus spots of the spatially distributed x-ray source array with programmable photon flux and pulse sequence for synchronization of x-ray exposure with data collection of the one or more x-ray detector arrays; and
the system being adapted to process and reconstruct collected images to form a three-dimensional reconstructed image of an object and to display the image in an image display apparatus.

14. A stationary gantry x-ray computed tomography imaging system, comprising multiple and substantially parallel imaging planes, the system comprising:
multiple imaging planes, wherein each imaging plane comprises one or more linear spatially distributed field emission x-ray source arrays and opposing x-ray detector arrays that are arranged in a square or polygon geometry, wherein the orientation of the x-ray source arrays and detector arrays in each imaging plane are off-set from each other to provide increased angular coverage;
an electronic control for controlling x-rays from individual x-ray focus spots of the one or more spatially distributed x-ray source arrays with programmable photon flux and pulse sequence for synchronization of x-ray exposure with data collection of the one or more x-ray detector arrays; and
the system being adapted to process and reconstruct collected images to form a three-dimensional reconstructed image of an object and to display the image in an image display apparatus.

15. A stationary non-rotating gantry computed tomography imaging system for imaging of head, neck and spine, the system comprising:
one or more distributed carbon nanotube field emission x-ray source arrays adapted to generate multiple x-ray beams from different projection angles;
one or more x-ray detector arrays arranged substantially opposing the x-ray source arrays adapted to detect the x-ray radiation;
an electronic control unit for controlling the x-ray from individual x-ray focus spots with programmable photon flux and pulse sequence for synchronization of x-ray exposure with data collection of the x-ray detector arrays;
an imaging processing unit for 3D image reconstruction; and
a wireless device for transmission of acquired images or reconstructed images to a remote interpretation station.

16. The system of claim 15, comprising:
multiple linear carbon nanotube field emission x-ray source arrays adapted to generate multiple x-ray beams from different projection angles in a sequential pattern;
multiple x-ray detector arrays arranged substantially opposing the x-ray source arrays and adapted to record x-ray radiation from the x-ray arrays;
an electronic control unit for controlling the x-ray from individual x-ray focus spots with programmable photon flux and pulse sequence for synchronization of x-ray exposure with data collection of the x-ray detector arrays;
an imaging processing unit using iterative reconstruction algorithm for 3D image reconstruction; and
a wireless device for transmission of acquired images or reconstructed images to a remote interpretation station.

17. A method of operating an imaging system, the method comprising:
configuring a stationary distributed x-ray source array to emit x-ray from one or more individually addressable focus spots;
positioning an object to be imaged between the distributed x-ray source array and at least one x-ray detector;
providing an electronic control unit to control sequence and x-ray parameter of one or more individual x-ray beams from the x-ray source array;
detecting the x-ray emitted from the x-ray source array;
generating a visualization of one or more images of the object based on x-rays detected by the at least one x-ray detector;
processing the one or more images to form a 3-dimensional reconstructed image of the object; and
generating a visualization of the image in 2D or 3D in a display unit.

18. The method of claim 17, wherein the electronic control unit synchronizes image acquisition with physiological signals of the object.

19. The method of claim 17 comprising analyzing the generated visualization and altering one or more image acquisition parameters based on analysis of the generated visualization to generate an improved image.

20. The method of claim 17, wherein the one or more image acquisition parameters is selected from a group consisting of: the number of projection views, the distribution of the project views, the keV and mAs from individual x-ray focus spots, the focus spot size, and the filtration of x-ray spectrum.

21. The method of claim 17, wherein the multiple energy CT images can be collected by either using detector arrays adapted for detecting x-ray energy from one or more energy bins or rapidly switching the x-ray energy of the source array between multiple x-ray energy levels;
